(19) United States
(12) Patent Application Publication
(10) Pub. No.: US 2019/0374165 A1
(43) Pub. Date: Dec. 12, 2019

(54) IMAGE PROCESSING APPARATUS AND METHOD

(71) Applicant: Canon Medical Systems Corporation, Otawara-shi (JP)

(72) Inventors: Ian POOLE, Edinburgh (GB); Satoshi MATSUNAGA, Nasushiobara (JP)

(73) Assignee: Canon Medical Systems Corporation, Otawara-shi (JP)

(21) Appl. No.: 16/001,981

(22) Filed: Jun. 7, 2018

(51) Int. Cl. A61B 5/00; A61B 8/08; G06T 7/11; A61B 8/00; G06N 3/08; G16H 30/20

(52) U.S. Cl. CPC A61B 5/7267; A61B 8/52; G06T 7/11; A61B 8/465; G06T 2207/30004; G06N 3/08; G16H 30/20; G06T 2207/10132; G06T 2207/20081; A61B 8/469

(57) ABSTRACT

An ultrasound diagnosis apparatus comprises processing circuitry configured to set initial values for a set of imaging parameters for use in acquiring ultrasound data for an ultrasound image, the set of imaging parameters comprising at least one acquisition parameter; acquire the ultrasound data according to the initial values for the set of imaging parameters, and process the ultrasound data to obtain the ultrasound image; extract imaging information in a region of interest of the ultrasound image; obtain predicted values for the set of imaging parameters using the extracted at least one feature and a machine learning algorithm trained using user-selected values for the set of imaging parameters, wherein the user-selected values are selected to provide a preferred appearance of the region of interest; and set the predicted values for the set of imaging parameters for use in acquiring further ultrasound data for a further ultrasound image.

### FIELD

Embodiments described herein relate generally to a method of, and apparatus for, image processing, for example a method of optimizing acquisition parameters in ultrasound imaging.

### BACKGROUND

It is known to acquire image data using ultrasound. An ultrasound (UL) acquisition is typically controlled by a large number of parameters. The parameters that control the ultrasound acquisition may comprise hardware parameters, which may also be referred to as acquisition parameters.

Hardware parameters may include, for example, parameters relating to frequency, pulse duration, pulse power, frame rate, depth and focus (for example, F-number). When a change is made to a value for a hardware parameter, ultrasound data may be re-acquired with the new value for the hardware parameter. The re-acquisition may take some time to acquire, for example around 300 ms.

Software parameters may be used in processing ultrasound data to form an ultrasound image. Software parameters may include, for example, parameters relating to dynamic range, gain, gamma correction, or filter setting. Software parameters may also be referred to as post-processing parameters. When a change is made to a value for a software parameter, image data may be post-processed with the new value for the software parameter without performing a new ultrasound image data acquisition. In some circumstances, software post-processing changes may be effected very rapidly.

Hardware parameters and software parameters may also be referred to as imaging parameters.

Parameter values that provide a good quality image (for example, an image that provides a clear view of an anatomical region of interest) may be referred to as optimal or ideal settings. Optimal settings for the various software and hardware parameters may depend on various factors. For example, optimal settings for some parameters may depend on the anatomy to be imaged. Optimal settings for some parameters may depend on the size of the patient. A quantity of intervening fat may have a particular effect on the optimal settings for a given acquisition. There may also be a degree of sonographer preference in a choice of optimal parameter settings.

Manual adjustment of acquisition parameters may be time consuming even for an experienced sonographer. Manual adjustment of acquisition parameters may be challenging for a less experienced user.

Many ultrasound machines incorporate a limited amount of automation. For example, some machines manipulate gain and time gain compensation (TGC). However, a change in hardware parameters may take significant time to be effected. Therefore, in some circumstances only a limited number of trials of hardware parameters may be possible. For example, a sonographer may choose to adjust values for only a small number of parameters and/or to adjust values for the parameters only a small number of times, in order to obtain a final ultrasound image within an acceptable timescale.

### BRIEF DESCRIPTION OF THE DRAWINGS

Embodiments are now described, by way of non-limiting example, and are illustrated in the following figures, in which:—FIG. 1 is a schematic illustration of an apparatus in accordance with an embodiment;

FIG. 2 is a flow chart illustrating in overview a training method in accordance with an embodiment;

FIG. 3 is a schematic illustration of a display screen and a control screen of an apparatus in accordance with an embodiment;

FIG. 4 is a flow chart illustrating in overview a machine learning method in accordance with an embodiment;

FIG. 5 is a flow chart illustrating in overview a parameter prediction method in accordance with an embodiment; and

FIG. 6 is a plot of parameter convergence.

### DETAILED DESCRIPTION

Certain embodiments provide an ultrasound diagnosis apparatus comprising processing circuitry configured to set initial values for a set of imaging parameters for use in acquiring ultrasound data for an ultrasound image, the set of imaging parameters comprising at least one acquisition parameter; acquire the ultrasound data according to the initial values for the set of imaging parameters, and process the ultrasound data to obtain the ultrasound image; extract imaging information in a region of interest of the ultrasound image; obtain predicted values for the set of imaging parameters using the extracted at least one feature and a machine learning algorithm trained using user-selected values for the set of imaging parameters, wherein the user-selected values are selected to provide a preferred appearance of the region of interest; and set the predicted values for the set of imaging parameters for use in acquiring further ultrasound data for a further ultrasound image.

Certain embodiments provide a training apparatus for training a machine learning algorithm to predict values for a set of imaging parameters, the training apparatus comprising processing circuitry configured to: for each of a plurality of anatomical regions of a plurality of subjects, obtain a user-selected set of values for a set of imaging parameters, wherein the user-selected set of values is selected by the user as providing a preferred appearance of the anatomical region of the subject in an ultrasound image; obtain training samples for the plurality of anatomical regions of the plurality of subjects, each training sample comprising a respective set of values for the imaging parameters and an ultrasound image acquired by scanning the anatomical region of the subject using said respective set of values; and train the machine learning algorithm using the training samples and the user-selected sets of values, such that the machine learning algorithm is configured to receive initial values for the imaging parameters and at least part of an ultrasound image obtained using the initial set of values, and to output predicted values for the imaging parameters.

Certain embodiments provide a method for training a machine learning algorithm to predict values for a set of imaging parameters, the method comprising, for each of a plurality of subjects, for at least one anatomical region of the subject, using a medical scanner to scan the anatomical region of the subject to obtain ultrasound data using at least one user-supplied set of values for a set of imaging parameters of the scanner, the set of imaging parameters comprising at least one acquisition parameter; processing the ultrasound data to obtain an ultrasound image; receiving a user-selected set of values that are selected by the user as providing a preferred appearance of the anatomical region of the subject in the ultrasound image; automatically generating a plurality of sets of values for the set of imaging parameters; using the medical scanner to scan the anatomical region of the subject using each of the automatically-generated sets of values for the set of imaging parameters, thereby to obtain for each of the automatically-generated sets of values a respective training sample, the training sample comprising the automatically-generated set of values and an ultrasound image obtained from the scanning of the anatomical region of the subject using the automatically-generated set of values; and training a machine learning algorithm based on the training samples and user-selected sets of values, such that the machine-learning algorithm is configured to receive initial values for the imaging parameters and an ultrasound image obtained using the initial values, and to output predicted values for the imaging parameters.

An apparatus 10 according to an embodiment is illustrated schematically in FIG. 1. The apparatus 10 is configured to acquire ultrasound data from an ultrasound scan and to process the ultrasound data to obtain an ultrasound image.

In the present embodiment, the apparatus 10 is also configured to train a machine learning algorithm to adjust values for a plurality of ultrasound imaging parameters. In other embodiments, the training of the machine learning algorithm may be performed by a separate computing apparatus, for example a PC or workstation (not shown).

In the present embodiment, the apparatus 10 comprises an ultrasound machine 12 and associated measurement probe 14. The ultrasound machine 12 may also be referred to as an ultrasound diagnosis apparatus. Any suitable type of ultrasound machine 12 and measurement probe 14 may be used. In other embodiments the medical diagnostic apparatus 10 may comprise a scanner apparatus of an alternative modality.

The ultrasound machine 12 comprises a main display screen 16 for displaying a main ultrasound image. The ultrasound machine 12 further comprises a scanner console 20. The scanner console 20 comprises a control screen 18 for displaying control information and input devices comprising various control knobs 19. The input devices may further comprise a computer keyboard, a mouse or a trackball (not shown). The control knobs 19 and/or other input devices may be used to adjust values for a plurality of hardware and software parameters. In the present embodiment, the control screen 18 is a touch screen, which is both a display device and a user input device. Further embodiments may comprise a control screen 18, display screen or main display screen 16 that does not form part of the ultrasound machine 12. The ultrasound machine 12 also comprises a data store 30.

The ultrasound machine 12 comprises a processing apparatus 22 for processing of data, including image data. The processing apparatus 22 comprises a Central Processing Unit (CPU) and Graphical Processing Unit (GPU). The processing apparatus 22 includes acquisition circuitry 24, training circuitry 26, and machine learning circuitry 28. The acquisition circuitry 24, training circuitry 26, and machine learning circuitry 28 may each be implemented in the CPU, in the GPU, or in a combination of the CPU and the GPU.

In the present embodiment, the various circuitries are each implemented in the CPU and/or GPU of processing apparatus 22 by means of a computer program having computer-readable instructions that are executable to perform the method of the embodiment. However, in other embodiments each circuitry may be implemented in software, hardware or any suitable combination of hardware and software. In some embodiments, the various circuitries may be implemented as one or more ASICs (application specific integrated circuits) or FPGAs (field programmable gate arrays).

In alternative embodiments the processing apparatus 22 comprising the acquisition circuitry 24, training circuitry 26, and machine learning circuitry 28 may be part of any suitable medical diagnostic apparatus (for example a CT scanner or MR scanner) or image processing apparatus (for example, a PC or workstation). The processing apparatus 22 may be configured to process any appropriate modality of imaging data.

In some embodiments, different circuitries are implemented in different apparatuses. For example, in some embodiments, the machine learning circuitry 28 is implemented in a computing apparatus, for example a PC or workstation, that does not form part of the ultrasound machine 12.

The processing apparatus 22 also includes a hard drive and other components including RAM, ROM, a data bus, an operating system including various device drivers, and hardware devices including a graphics card. Such components are not shown in FIG. 1 for clarity.

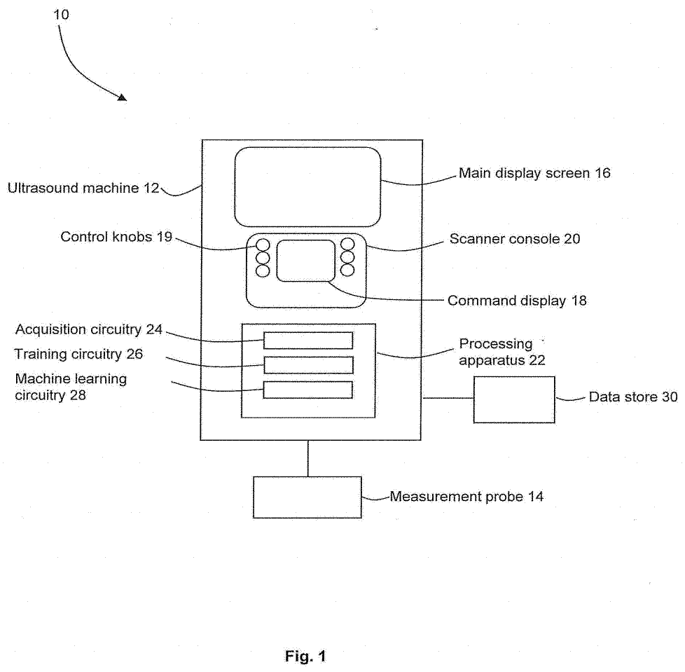

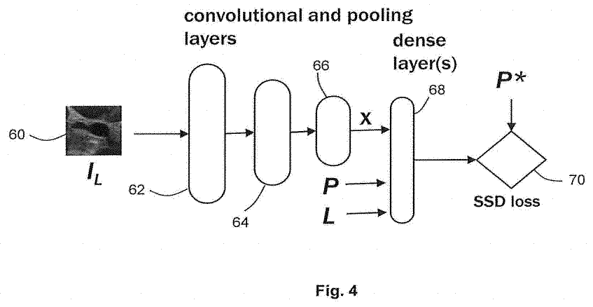

The system of FIG. 1 is configured to perform a training process having a series of stages as illustrated in overview in the flow chart of FIG. 2, and an image optimization process having a series of stages as illustrated in overview in the flow chart of FIG. 5. In further embodiments, the process of FIG. 2 may be performed by a first apparatus and the process of FIG. 5 may be performed by a second, different apparatus.

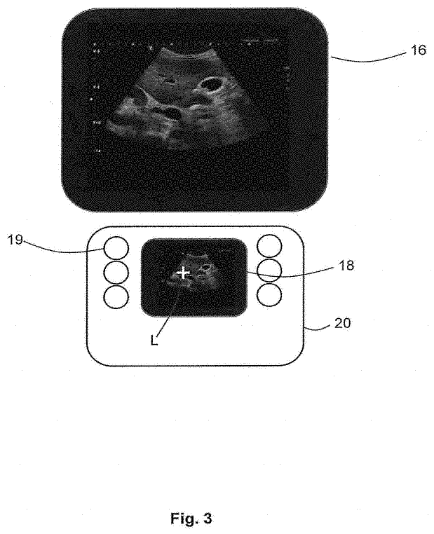

We first describe the training process of FIG. 2. The training process of FIG. 2 is used to train a machine learning algorithm to predict a set of parameter values that may provide desired image properties. In the present embodiment, the machine learning algorithm comprises a feature-based machine learning algorithm. In other embodiments (including that illustrated in FIG. 4), the machine learning algorithm comprises a neural network, for example a convolutional neural network. In further embodiments, any image-based machine learning algorithm may be used. For example, extraction of texture features may be followed by a support vector machine (SVM), k-nearest neighbor (k-NN), or decision forest algorithm.

The training process of FIG. 2 comprises obtaining repeated ultrasound acquisitions from each of a plurality of different human subjects, and training the machine learning algorithm using data from the repeated ultrasound acquisitions.

In the present embodiment, 20 human subjects are used in the training process. The human subjects are chosen such that they cover a range of patient sizes. In particular, the human subjects are chosen such that they have different amounts of fat. In other embodiments, any suitable number of human or animal subjects may be used in the training process.

At stage 40 of FIG. 2, the training process starts with a first human subject. Medical data related to the first human subject (for example, subject height, weight and gender) may be obtained and recorded.

At stage 42, a sonographer selects a first anatomical region of the subject. In the present embodiment, 10 anatomical regions of interest have been identified for scanning. The sonographer selects a first one of those 10 anatomical regions of interest, for example the aortic valve. Each anatomical region may comprise, for example, at least part of an organ, a bone or a vessel. The anatomical regions may comprise, for example, the carotid artery, the liver, or the kidneys.

The sonographer positions the transducer probe 14 in an appropriate location for imaging the first anatomical region (in this example, the aortic valve).

At stage 44, the sonographer manually adjusts values for at least some of a set of ultrasound imaging parameters to obtain a preferred image. The sonographer may also adjust a position and/or orientation of the transducer probe 14. The sonographer may adjust parameter values iteratively until a preferred image is obtained. New ultrasound data is acquired by the ultrasound machine 12 each time the sonographer changes a hardware parameter value, and the new ultrasound data is processed to obtain a new ultrasound image. Each acquisition of ultrasound data may take, for example, 300 ms. In some circumstances, the changing of a software parameter value may not involve a new ultrasound data acquisition. Instead, the displayed ultrasound image may be updated to take account of the new software value without a new acquisition being performed.

The process of adjusting parameter values that is performed by the sonographer at stage 44 may be similar to a process of adjusting parameters that is performed by the sonographer in normal, routine use of the ultrasound machine 12. Alternatively, in some embodiments, the sonographer may perform a more lengthy and/or precise adjustment of parameter values than is performed in normal use. The process of adjusting parameters may be described as a manual optimization of parameters by the sonographer.

In the present embodiment, the set of ultrasound parameters comprises parameters that are adjustable by the sonographer in normal use of the ultrasound machine 12. The sonographer may adjust values for any one or more of the set of ultrasound parameters.

In the present embodiment, the set of ultrasound parameters comprises both hardware parameters and software parameters. The hardware parameters comprise parameters relating to, for example, wave profile parameters, frequency parameters (for example, a frequency at which ultrasound is transmitted and/or a frequency at which ultrasound is received), pulse duration, pulse power, frame rate, depth and focus (for example, F-number). The software parameters comprise parameters relating to, for example, dynamic range, gain, gamma correction, and filter setting. In other embodiments, the set of ultrasound parameters may comprise only hardware parameters or only software parameters.

The adjusting of the parameter values by the sonographer may be in order to obtain a best possible diagnostic quality at a location L, where the location L is a location of the anatomical region of interest that was selected at stage 42 (for example, the aortic valve). The image that is preferred by the sonographer may be the image that has the best (for example, clearest) appearance of the anatomical region of interest.

In the present embodiment, location L is expressed as a set of coordinates that indicate the location of the anatomical region of interest in the two-dimensional image space of an ultrasound image.

As the sonographer adjusts the parameter values, the sonographer views an ultrasound image that is displayed on the main display screen 16. The image displayed on the main display screen 16 may be described as a current acquisition. The image displayed on the main display screen 16 is updated over time. The image displayed on the main display screen 16 is also updated for each change of parameter values.

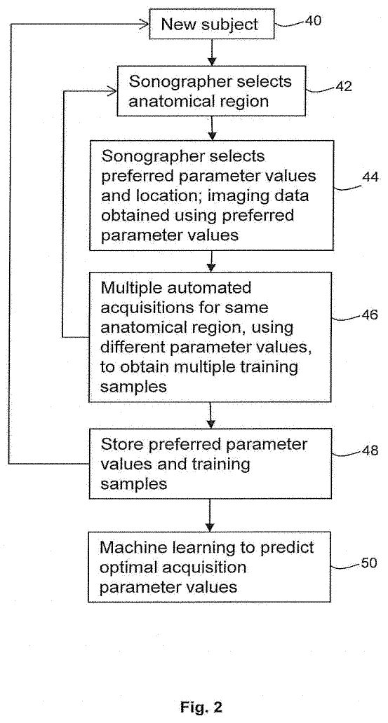

In the present embodiment, the same ultrasound image is displayed on the control screen 18 as is displayed on the main display screen 16. It may be said that the current acquisition shown in the upper main display is mirrored at a smaller scale in the lower control screen 18. The display of the same image on the main display screen 16 and the control screen 18 is illustrated in FIG. 3. In the present embodiment, the control screen 18 is touch-sensitive. In other embodiments, the main display screen 16 may be touch-sensitive.

When the sonographer has adjusted the parameter values to obtain a preferred image, the sonographer touches a point in the mirrored view of the control screen 18 to indicate the image region of clinical interest. In other embodiments in which the main display screen 16 is touch-sensitive, the sonographer may touch a point on the main display screen 16 to indicate the image region of clinical interest.

The point on the control screen that the sonographer touches is taken to be the location L of the anatomical region of interest in that image by touching the control screen 18. In FIG. 3, the location L is illustrated by a cross on the control screen 18.

The acquisition circuitry 24 records a set of coordinate values for the point L in the center of the cross.

In other embodiments, the sonographer selects a region of coordinate space, for example by drawing a bounding box around the anatomical region of interest, and the acquisition circuitry 24 records an extent of the region of coordinate space. In further embodiments, any method may be used for selecting a point or region for the location L of the anatomical region of interest. In some embodiments, the location L of the anatomical region of interest is detected automatically.

In the present embodiment, the image on which the sonographer touches the screen to provide the location L is taken to be the sonographer's preferred image. In other embodiments, the sonographer (or another user) may choose a preferred image in any suitable manner, for example using any suitable controls.

For the sonographer's preferred image, the acquisition circuitry 24 obtains:
a preferred ultrasound image I*
the set of parameter values P* with which the preferred image is obtained.

The ultrasound image I* may be referred to as an optimal acquired image. The set of parameter values P* may be referred to as a set of optimal acquisition parameters. We note that in this context the term optimal is used to refer to the image and parameters that the sonographer has considered, in their own subjective view, to be the preferred image and parameters. In practice, it may be the case that different sonographers may identify different images and parameters as being optimal, or that the same sonographer may identify different images and parameters as being optimal if the manual optimization process were to be conducted repeatedly.

In the present embodiment, one ultrasound image I* and set of parameter values P* is obtained at stage 44. In other embodiments, more than one ultrasound image I* and set of parameter values P* is obtained at stage 44. Several (for example, three) manually optimized acquisitions may be obtained by independent repeat. For example, parameter values may be reset to default values between repeats of the manual acquisition process described above. The default values may comprise standard preset parameters.

Once the preferred ultrasound image(s) I* and set(s) of parameter values P* for the sonographer's preferred image(s) have been obtained, the process of FIG. 2 moves on to stage 46.

At stage 46, the training circuitry 26 automatically generates multiple different sets of parameter values for the set of ultrasound parameters.

In the present embodiment, the training circuitry 26 generates 200 sets of parameter values P. The parameter values are randomly chosen within predetermined limits, with uniform sampling density. For example, predetermined limits for each parameter may be representative of a range of values for that parameter in normal use of the ultrasound machine.

In other embodiments, any suitable sampling strategy may be used to generate the sets of parameter values P. In some embodiments, the sampling strategy comprises sampling on a pre-determined regular grid, within limits for each parameter. In other embodiments, the sampling strategy comprises random uniform sampling within limits for each parameter. In further embodiments, the sampling strategy comprises sampling a random Gaussian (normal) distribution having a mean at a parameter value preferred by a sonographer. The sampling distribution may be set with reference to parameter limits.

Clamping may be imposed on values for one or more of the parameters to ensure that safe limits on the parameter are not exceeded. For example, clamping may be used to ensure that safe limits on transducer power are not exceeded. Some of the limits may depend on parameter combinations. For example, a safe value for a first parameter may be dependent on a value for a second parameter and/or values for further parameters.

Any suitable number of sets of parameter values P may be generated.

Each of the 200 sets of parameter values P differs from each other set of parameter values P by a value of at least one parameter.

The acquisition circuitry 24 causes the ultrasound machine 12 to acquire a respective set of ultrasound data using each of the sets of parameter values P, and to process the set of ultrasound data to obtain an ultrasound image I for the set of parameter values P. We note that references to operations that are performed on an ultrasound image I may refer to operations that are performed on a data set comprising data values (for example, intensity values) for elements (for example, pixels) of the ultrasound image.

In the present embodiment, all 200 ultrasound images are acquired with the transducer probe 14 held in substantially the same position and orientation, which are the same as for the sonographer's preferred image at stage 44. In other embodiments, different transducer probe positions may be used.

The acquisition of ultrasound images at stage 46 is automated and does not require input from the sonographer. Since the parameter values are adjusted automatically, the adjustment of the parameter values and the acquisition of the ultrasound image for each set of parameter values may be performed rapidly. For example, a new ultrasound image may be acquired every 300 ms. Although we refer to the acquisition of ultrasound images, it may be the case that the ultrasound images are never displayed (for example on the display screen). Data representing the ultrasound images may be stored without the images being displayed.

For each of the 200 sets of parameter values P, the acquisition circuitry 24 obtains:
the set of parameter values P,
an ultrasound image I that was acquired using the set of parameter values P,
the location L for the anatomical region.

In the present embodiment, the location L for the anatomical region is the same location L that was selected by the sonographer in stage 44. In other embodiments, any method of determining the location L may be used.

A set of data comprising an ultrasound image I, the corresponding location L of the anatomical region, and the corresponding set of parameter values P may be referred to as a set of training data, or may be referred to as a training sample (I,L,P).

All of the 200 training samples (I,L,P) obtained at stage 46 are acquired by imaging the same anatomical region of the same subject (for example, the subject's aortic valve) using different sets of parameter values.

After stage 46, the process of FIG. 2 returns to stage 42 and the sonographer selects a second, different anatomical region of the first subject, for example the carotid artery of the first subject. The process then proceeds again to stage 44, at which the sonographer performs a manual optimization of parameter values for that second anatomical region. The acquisition circuitry 24 obtains the sonographer's preferred parameter values P* for the second anatomical region and an ultrasound image I* obtained with those preferred parameter values. The sonographer also indicates a location L for the second anatomical region.

At stage 46, the training circuitry 26 generates a further 200 sets of parameter values P. The parameter values are randomly chosen within the same predetermined limits as were used for the first anatomical region, with uniform sampling density. In other embodiments, the same sets of parameter values are used for the second anatomical region as were used for the first anatomical region. For each of the further sets of parameter values P, the acquisition circuitry 24 obtains a respective training sample (I,L,P) by scanning the second anatomical region using the set of parameter values P.

Stages 42 to 46 are repeated until data has been obtained for all 10 anatomical regions of the first subject.

At stage 48, the acquisition circuitry 24 stores in the data store 30 the sonographer's preferred set of parameter values P*, the ultrasound image I*, and the 200 training samples (I,L,P) that were obtained for each of the 10 anatomical regions of the first subject. In other embodiments, the ultrasound images I* obtained using the preferred sets of parameter values P* may not be stored. In further embodiments, the data may be stored in any suitable data store, for example in a data store forming part of a PACS.

The process of FIG. 2 then returns to stage 40 and a second subject is selected.

Stages 40 to 48 are repeated until data has been obtained for each of the 20 different subjects.

In the present embodiment, the same 10 anatomical regions are scanned for each of the subjects. Different sets of parameter values are generated for each anatomical region of each subject. In other embodiments, the same sets of parameter values may be used for different regions and/or different subjects.

The acquisition of preferred parameter values P* may be described as ground truth collection. The scheme of ground truth collection and training data collection described above may be described as being efficient. Although 200 training images are acquired at each anatomical location, sonographer interaction is involved in only the one optimal image P. The collection of ground truth may be less onerous than would be the case if the sonographer were to assess the quality of each of the sets of training data.

Stages 40 to 48 may be described by the following algorithm, expressed as pseudocode:

Overall training data collection:

```
data = [ ]
for N patients:
  for k anatomical regions:
    P* = ideal sonographer settings
    I* = acquire (P*)
    L = location of interest
    for m acquisitions:
      P = random parameters
      I = acquire (P)
      append (L, P, I, P*, I*) to data
```

We have described specific numbers of subjects, anatomical regions and sets of parameter values merely as an example. Any suitable numbers of subjects, anatomical regions and/or sets of parameter values may be used in practice. For example, the number of sets of parameter values for each anatomical region of each subject may be at least 10, optionally at least 50, further optionally at least 100. The number of subjects may be at least 5, optionally at least 10, further optionally at least 50. The number of parameters in the set of parameters may be at least 5, optionally at least 10. The subjects may be human or animal.

In the embodiment described above, the ultrasound parameters that are varied at stage 46 comprise both hardware parameters and software parameters. Software parameters are varied, and images acquired using those parameters are saved, at the point of acquisition.

In other embodiments, only hardware parameters are varied at stage 46. Software parameters are varied, and new images using the software parameters are saved, after all of the acquisitions with different hardware parameters have been made. In such embodiments, values for the software parameters with which the acquisitions were made may be varied without having to retake the acquisitions.

In some embodiments, software parameters may be varied and images saved offline, for example prior to running a machine learning algorithm as described below.

At stage 50, the machine learning circuitry 28 receives the sets of preferred parameter values P*, sets of image data I*, and training samples (I,L,P) that have been stored in the data store 30 for each of the 20 subjects.

In the present embodiment, the number of anatomical regions is 10, the number of subjects is 20, and the number of acquisitions per anatomical region per subject is 200. Therefore, 40,000 training samples are available to the machine learning circuitry 28.

Although a particular ordering of acquisitions is described above, in other embodiments acquisitions may be performed in any suitable order. For example, the automatic acquisition of the training samples may be performed before the acquisition of the sonographer's preferred image (if the sampling method used does not depend on the parameter values of the sonographer's preferred image).

Each set of preferred parameter values P* may be referred to as an optimal parameter setting. In the present embodiment, 200 optimal parameter settings P* are available to the machine learning circuitry 28. Each optimal parameter setting P* is associated with 200 training samples acquired for the same subject and same anatomical region.

The number of training samples may be considered to be plentiful for a machine learning approach to the prediction of optimal parameter settings P* using training samples (I,L,P).

In the present embodiment, the machine learning circuitry 28 performs a machine learning process to train a machine learning algorithm using a feature based approach. The machine learning algorithm may also be referred to as a predictor.

For each training sample (I,L,P), the machine learning circuitry 28 selects a region of interest in the image data set I that is centred on the location L. The region of interest may be referred to as I_{L}. The dimensions of the region of interest may be predetermined. For example, the machine learning circuitry 28 may select a region of interest I_{L} having a predetermined height and width in centimeters.

In the present embodiment, the region of interest I_{L} is a 5 cm×5 cm region of the image data I that is centred on the location L of the anatomical region of interest.

The machine learning circuitry 28 extracts imaging information from the region of interest I_{L}. For example, the imaging information may comprise a respective pixel value for each pixel in the region of interest I_{L}.

The machine learning circuitry 28 extracts a set of features from the imaging information. In the present embodiment, the set of features includes at least one intensity distribution and a plurality of texture features. In other embodiments, the machine learning circuitry 28 may extract any suitable features from the extracted region I_{L}, for example intensity, gradient, texture or SURF features (Speeded Up Robust Features) for any position within the extracted region I_{L}.

The machine learning circuitry 28 uses a machine learning training algorithm to distinguish which of the extracted features are the best features for predicting values for the set of acquisition parameters. In the present embodiment, the machine learning circuitry selects a set of features x comprising at least one intensity distribution and at least one texture feature. The set of parameter values P may be expressed as a vector. The set of features x is appended to the location L and the set of parameter values P to obtain a feature vector X=(x,L,P).

The machine learning circuitry 28 trains a regression method to predict preferred parameters P* based on feature vectors X=(x,L,P). In the present embodiment, the regression method comprises a support vector machine regression method. In other embodiments, the regression method comprises a decision forest regression method. In further embodiments, any suitable regression method may be used, for example an SVM (support vector machine), decision forest, K-nearest neighbors (Knn), linear model or logistic regression method. Any form of feature-based machine learning may be used that may be configured for regression.

The output of stage 50 is a machine learning algorithm that is trained to predict preferred parameters P*. The application of the machine learning algorithm is described below with reference to FIG. 5.

In a further embodiment, a convolutional neural network (CNN) approach is used at stage 50. The convolutional neural network approach is illustrated in the flow chart of FIG. 4. The CNN approach may be described as a deep learning approach. In other embodiments, any type of deep learning may be used. The deep learning CNNs used in the present embodiment may be, for example, as described in chapter 2 of “Deep Learning for Medical Image Analysis”, S. Kevin Zhou et al., Academic Press or chapter 9 of ‘Deep Learning’ by Ian Goodfellow, Yoshua Bengio and Aaron Courville, MIT Press

At stage 60 of FIG. 4, the machine learning circuitry 28 extracts a region of interest I_{L} from each training sample (I,L,P). For example, the region of interest may be a 5 cm×5 cm region of the image data I that is centered on the location L of the anatomical region of interest. The machine learning circuitry 28 extracts imaging information for the region of interest I_{L}. In the present embodiment, the imaging information comprises a respective pixel value for each pixel in the region of interest I_{L}.

Image pixel values for each region of interest I_{L} are input into multiple convolution and pooling layers, which are represented in FIG. 4 by layers 62, 64, 66. In practice, many more convolution and pooling layers may be used. The convolution layers maintain spatial information. The pooling layers have an output that is reduced in size when compared with their input.

The output of layer 66 is an image that is smaller than the extracted region of interest I_{L} that was the original input to the set of convolution and pooling layers. The smaller image is denoted by x. The smaller image x may be, for example, a 16×16 image.

The output x of layer 66 is passed into a dense layer 68. The dense layer flattens out the output x to provide a vector, for example a vector of 256 data items. At least some of the parameter values P are also added to the dense layer. For example, 6 or 7 data items from the parameter values P may be added. The dense layer therefore provides a long feature vector. In other embodiments, the location L may also be provided to the dense layer.

The output of the dense layer 68 is input to a sum of squared difference (SSD) stage 70. The optimal parameter setting P* is also input into the SSD 70. The sum of squared difference stage outputs a sum of squared difference of the input to the SSD stage, which may be the same or similar to the squared L2 norm of the input.

The machine learning circuitry 28 trains parameters of the various layers (for example, weights) using the training samples and ground truth. The training may be performed by any suitable method. In the present embodiment, the training is performed by back-propagation with stochastic gradient descent. The features that are used in prediction are discovered by back propagation.

As described above, there are two possible approaches to the training of the machine learning algorithm. In feature based machine learning embodiments, features (for example, texture features) are fed into, for example, an SVM, decision forest, Knn or linear model, any of which may be configured for regression. In deep learning embodiments, pixel values of the ultrasound image (which may be described as raw pixel values) are used directly. In the embodiment above, the deep learning method is the CNN. In other embodiments, decision forests may be used. In further embodiments, any suitable deep learning approach may be used.

In the embodiments described above, the machine learning algorithm is trained on all of the training data, which comprises images of 10 different anatomical locations of interest. By training the machine learning algorithm on a range of anatomies, the machine learning algorithm may also be able to be used on other anatomies that were not included in the training set. The machine learning algorithm may favor image characteristics that correspond to good image quality in a range of anatomies (for example, good contrast and/or good edge definition). A single machine learning algorithm may be used for a wide range of different anatomies.

By using a single machine learning algorithm on a wide range of anatomies, the sonographer does not have to declare which anatomy is currently be viewed in order to obtain predicted parameter values. Cross-anatomy training may improve predictions for all anatomies. The training of the machine learning algorithm may be considered to be a form of multi-task learning.

In other embodiments, separate machine learning algorithms may be trained for different anatomical locations of interest. For example, one machine learning algorithm may be trained for use in imaging the heart, and another machine learning algorithm may be trained for use in fetal imaging.

The training methods described above learn to predict the best values for ultrasound parameters directly. There is no intervening stage where image quality is quantified. The training samples are not assessed for quality. Instead, the machine learning algorithm learns which type of images are preferred from the sonographer's preferred parameter values.

Ground truth collection may be considerably simplified since the sonographer does not have to provide any quality assessment, for example any quality score. The sonographer merely selects what they consider to be the best image.

FIG. 5 is a flow chart illustrating in overview a method of an embodiment. In the process of FIG. 5, a learned predictor is used to predict parameter values for an ultrasound acquisition. In the present embodiment, the learned predictor has been trained using the method of FIG. 2. In other embodiments, any method of training the predictor may be used.

At stage 80 of FIG. 5, the acquisition circuitry 24 receives default settings P. The default settings comprise a respective value for each of the set of ultrasound parameters. In some embodiments, the default settings P are provided by a sonographer. The default settings may comprise preset values, for examples values that may be used as a preset during normal use of the ultrasound machine 12. In some embodiments, the default settings P are stored by the acquisition circuitry 24. In some cases, different default settings may be used for different anatomical regions and/or for different characteristics of the subject to be scanned (for example, different patient sizes).

At stage 82, the sonographer positions the transducer probe 14 such that an image of a desired anatomical region is displayed on the main display screen 16. The same image is displayed on the control screen 18. The sonographer indicates in the image on the control screen 18 a location L of the anatomical region. For example, the sonographer may indicate the location L by touching the control screen, as described above with reference to FIG. 3.

At stage 84, the acquisition circuitry 24 instructs the ultrasound machine 12 to acquire an ultrasound image using the default settings P. The ultrasound machine 12 acquires a set of ultrasound data using the default settings and processes the ultrasound data to obtain the ultrasound image I. The acquisition circuitry 24 stores the ultrasound image I, user-identified location L, and parameter values P.

At stage 86, the acquisition circuitry 24 applies the machine learning algorithm to the ultrasound image I, user-identified location L, and parameter values P.

The acquisition circuitry 24 extracts imaging information for a region I_{L} surrounding the user-identified location L. In the present embodiment, the imaging information comprises pixel values.

In the present embodiment, the acquisition circuitry 24 obtains a set of features from the imaging information for the region I_{L}. The set of features comprises at least one intensity distribution and at least one texture feature. In other embodiments, any suitable features may be used.

The set of features obtained from the imaging information for the region I_{L} by the acquisition circuitry 24 may be a subset of the set of features x that were used in training the machine learning algorithm. For example, it may be found in training that only some of the features of the set of features x contribute to a prediction of parameter values.

The acquisition circuitry 24 inputs the extracted features into the trained machine learning algorithm. The acquisition circuitry 24 may also input at least some of the parameter values P into the trained machine learning algorithm. The features that are input into the trained machine learning algorithm are the features that were found in training to contribute to a prediction of parameter values.

The machine learning algorithm outputs a new set of parameter values P.

The machine learning algorithm may have been trained using any suitable feature-based training algorithm, for example a training algorithm as described above with reference to FIG. 2.

In further embodiments, the machine learning algorithm comprises a neural network that has been trained using any suitable training method, for example a training method as described above with reference to FIG. 4. The acquisition circuitry 24 supplies the imaging information (for example, pixel values) to the neural network. The acquisition circuitry 24 may also supply the parameter values P to the neural network. The neural network outputs a new set of parameter values P.

At stage 88, the acquisition circuitry 24 determines whether the parameter values P have converged. Any suitable method may be used to determine whether the parameter values P have converged. For example, the acquisition circuitry 24 may determine a difference between the initial value for each parameter, and the new value for the parameter. If no parameter value differs by more than a threshold value, the parameter values may be considered to have converged. In some embodiments, the process is deemed to have converged when the change to any parameter over a predetermined number of iterations (for example, over the last two iterations or three iterations) is less than a predetermined threshold value. In other embodiments, the process is deemed to have converged when a maximum number of iterations is reached.

In order to compare the differences in parameter values across different parameters, the parameter space is scaled. For example, a respective variance for each parameter may be estimated from the set of training samples. The parameter space may then be scaled to have unit variance in each dimension. Euclidean (L2) distances in the parameter space may then be considered when determining differences between sets of values of the parameters.

If the parameter values have not converged, the process of FIG. 5 returns to stage 84. The acquisition circuitry 84 instructs the ultrasound machine 12 to acquire a set of image data using the new values for the parameter values P that were generated by the machine learning algorithm.

At stage 86, the acquisition circuitry 24 uses the machine learning algorithm to predict a further set of parameter values P, using a method as described above in relation to the previous instance of stage 86.

At stage 88, the acquisition circuitry again determines whether the parameter values P have converged. If the parameter values P have not converged, the process returns to stage 84.

If at any instance of stage 88 the parameter values P are found to have converged, the process of FIG. 5 moves to stage 90. The current parameter values P are taken to be the preferred, or optimal, parameter values P* for the subject and anatomical region being scanned.

In the present embodiment, the optimization of the parameters using the method of FIG. 5 is completed within 2 seconds. Both display screens 16, 18 are updated accordingly. The acquisition circuitry 24 displays the set of imaging data acquired using the preferred parameter values P* on the main display screen 16 and control screen 18.

In the present embodiment, an undo function is provided to the user. The undo function (which may also be described as an undo facility) allows the user to return to the original default set of parameters instead of the preferred parameter values P* that have been obtained using the process of FIG. 5. In other embodiments, a function may be provided that allows the user to step back to any previous set of parameter values.

In the present embodiment, the sonographer may choose to perform a final manual adjustment to the preferred parameter values P*. For example, the sonographer who is performing the process of FIG. 2 may have different preferences than the sonographer or sonographers who provided the ground truth data.

The process of FIG. 5 may provide an iterative runtime acquisition parameter prediction. In the present embodiment, the process of FIG. 5 is performed automatically, except for the user identification of the location at stage 82. In the process of FIG. 5, the sonographer does not have to provide any input beyond touching the appropriate location.

The process of optimizing the parameter values P at stages 84 to 88 is performed in real time. Each ultrasound acquisition at stage 84 may take, for example, 300 ms. The prediction of stage 86 may be very fast. For example, the prediction stage may take less than 10 ms. In other embodiments, the prediction stage may take less than 100 ms, optionally less than 50 ms, further optionally less than 20 ms.

The optimization process may be performed in, for example, less than 1 second, less than 2 seconds, or less than 5 seconds. The optimization process may comprise several iterations of acquisition and prediction. The overall time taken for the optimization process may be dominated by the time taken for acquisition. The time taken for the prediction of stage 86 may be much less than the time taken for the acquisition of stage 84.

In some circumstances, the method of FIG. 5 may be used as part of a clinical examination without unduly delaying the clinical examination.

In some circumstances, the method of FIG. 5 may provide a faster and/or better optimization of ultrasound parameters than would be obtained through manual optimization by a sonographer. In some circumstances, using the method of FIG. 5 for parameter optimization may allow the sonographer to focus on other aspects of the ultrasound examination, for example clinical aspects.

By using the method of FIG. 5, optimal parameters P* may be found in a small number of iterations, for example in 5 or fewer iterations. It may be particularly desirable for the optimal parameters to be obtained in a small number of iterations when hardware parameters are involved. In order to obtain results with a change in hardware parameters, a new acquisition may need to be performed. It may be desirable to limit a number of acquisitions that is performed.

Runtime optimization using the method of FIG. 5 may be direct and feasible for many parameters. The method of FIG. 5 does not involve a gradient based optimization.

The method of FIG. 5 may be capable of rapidly optimizing multiple parameters. The method of FIG. 5 may be able to adapt to diverse anatomical locations (for example, if the machine learning algorithm is trained on a wide range of anatomical locations).

The method of FIG. 5 may provide a more comprehensive adjustment of parameters than may usually be provided by manual adjustment. The method of FIG. 5 may provide adjustment of a wider range of parameters than may usually be provided by manual adjustment. Unproductive examination time may be reduced. A lower proportion of the sonographer's time in the examination may be spent on the adjustment of parameters. A higher proportion of the sonographer's time may be spent on the clinical examination.

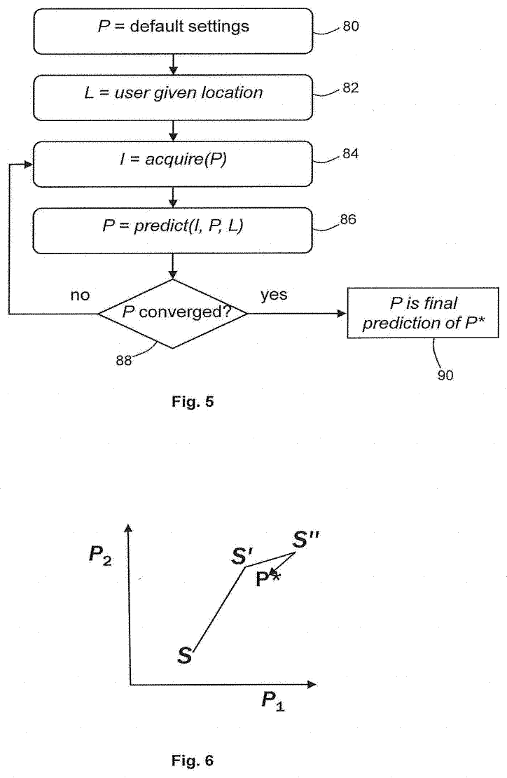

FIG. 6 is a schematic illustration of parameter adjustment using the process of FIG. 5, when values for only two parameters are adjusted (in practice, values for many more parameters may be adjusted).

Values for a first parameter P₁ are shown on the x axis of FIG. 6. Values for a second parameter P₂ are shown on the y axis.

The process of FIG. 5 starts with a default set of parameter values. The default set of parameter values are shown as point S on FIG. 6.

A set of image data I_{S} is obtained using the default set of parameter values S. The machine learning algorithm is applied to the set of image data I_{S}, default set of parameter values S and location L to obtain a new set of parameter values S′, which are shown by a further point on FIG. 6. It can be seen that the parameter values for both P₁ and P₂ are increased.

A set of image data I_{S′} is obtained using parameter values S′. The machine learning algorithm is then applied to image data I_{S′}, parameter values S′ and the location L to obtain a further set of parameter values S″.

A set of image data I_{S″} is obtained using parameter values S″. The machine learning algorithm is then applied to image data I_{S″}, parameter values S″ and the location L to obtain a final set of parameter values. The final set of parameter values are found to have converged and so are designated as an optimal set of parameters P*.

In some circumstances, a depth parameter may be treated differently from other parameters. A depth parameter may be determined directly from the user-provided location L, since the user indicates L on the display and the coordinate system of the display is known.

In the embodiments described above, a single machine learning algorithm is trained using 40,000 training samples that are obtained using the same ultrasound machine 12. In some cases, the ground truth data may all be obtained by the same sonographer.

In further embodiments, a machine learning algorithm trained on one ultrasound machine 12 may be used on other ultrasound machines, for example other ultrasound machines of the same model. The collection of training data and ground truth data and the learning of the optimal parameter prediction may be performed during development of an ultrasound machine, for example using a test machine.

In some embodiments, a new machine learning algorithm may be trained for each specific ultrasound machine. For example, collection of training data and/or ground truth data may be repeated at a customer site. The machine learning algorithm may be adapted to site preferences and/or individual sonographer preference. The efficiency of the ground truth collection may make such adaptation feasible.

Different sonographers may have different preferences with regard to image characteristics. It may be the case that different sonographers would decide on different optimal parameters for the same acquisition. In some embodiments, the machine learning algorithm is trained to the preferences of an individual sonographer.

In some embodiments, training the machine learning algorithm for a specific sonographer may comprise obtaining new ground truth parameter values from that sonographer. In other embodiments, an individual sonographer may selected a plurality of preferred images from a plurality of archived image. The machine learning algorithm may use the selected images to train or adapt the machine learning algorithm to the preferences of the sonographer.

References to acquisition of images above may comprise the acquisition (and, optionally, storage) of sets of image data. The acquisition of such sets of image data may comprise the processing of raw imaging data (for example, raw ultrasound data) to obtain image data that is representative of an image, for example an image for display. The processing of the raw imaging data may comprise, for example, reconstruction, pre-processing and/or filtering.

Methods described above may be used to predict values for parameters of any suitable medical imaging scanner. Methods described above may be used to predict values for any suitable hardware and/or software parameters. Methods described above may be applied to imaging of any suitable human and/or animal subjects.

In some embodiments, machine learning algorithms are trained that are specific to particular anatomical regions. In other embodiments, machine learning algorithms may be used that are not dependent on anatomical region.

Certain embodiments provide a medical imaging method for optimizing ultrasound acquisition parameters, by means of: a user provided location of interest; prediction by machine learning of best parameters from an acquired image, given location and current parameters; iterative application of the learned predictor during the clinical examination.

The machine learning algorithm may be based on intensity distribution and texture features measured from a region around the user provided location of interest. The machine learning algorithm may use a convolutional neural network to learn image features.

Image features, parameter values, location of interest may be combined by a classifier such as an SVM, decision forest, logistic regression. Image features, parameter values location of interest may be combined by a dense layer added to the convolutional neural network.

Training data may be collected by acquiring multiple images for each patient and anatomy, using randomized parameter values, accompanied by a single sonographer specified parameter set.

Certain embodiments provide an ultrasound diagnosis apparatus, comprising: processing circuitry configured to set application values of imaging parameters including an acquisition parameter for acquiring echo data on which ultrasound image is based; acquire the ultrasound image according to the application values; extract a texture feature from image information in a region of interest set in the ultrasound image; predict optimal values of the imaging parameters for optimizing the image information using the texture feature and a machine learning algorithm trained using acceptable values of the imaging parameters, the acceptable values being of when acceptable image information is acquired in the region of interest, and; set the optimal values as the application values. The acquisition parameter may comprise a wave profile parameter, a frequency of a transmitted ultrasound, or frequency of a received ultrasound. The processing circuitry may be configured to vectorize the application values, and; predict the optimal values further using the vectorized application value and a location of the region of interest. The processing circuitry may be configured to repeat a series of process from the setting of the application values to the predicting of the optimal values, two or more times. The ultrasound diagnosis apparatus may further comprise a touch command screen configured to display the ultrasound image, and; receive the setting of the region of interest in the displayed ultrasound image.

Whilst particular circuitries have been described herein, in alternative embodiments functionality of one or more of these circuitries can be provided by a single processing resource or other component, or functionality provided by a single circuitry can be provided by two or more processing resources or other components in combination. Reference to a single circuitry encompasses multiple components providing the functionality of that circuitry, whether or not such components are remote from one another, and reference to multiple circuitries encompasses a single component providing the functionality of those circuitries.

Whilst certain embodiments have been described, these embodiments have been presented by way of example only, and are not intended to limit the scope of the invention. Indeed the novel methods and systems described herein may be embodied in a variety of other forms; furthermore, various omissions, substitutions and changes in the form of the methods and systems described herein may be made without departing from the spirit of the invention. The accompanying claims and their equivalents are intended to cover such forms and modifications as would fall within the scope of the invention.

1. An ultrasound diagnosis apparatus comprising processing circuitry configured to:
set initial values for a set of imaging parameters for use in acquiring ultrasound data for an ultrasound image, the set of imaging parameters comprising at least one acquisition parameter;
acquire the ultrasound data according to the initial values for the set of imaging parameters, and process the ultrasound data to obtain the ultrasound image;
extract imaging information in a region of interest of the ultrasound image;
obtain predicted values for the set of imaging parameters using the imaging information and a machine learning algorithm trained using user-selected values for the set of imaging parameters, wherein the user-selected values are selected to provide a preferred appearance of the region of interest; and
set the predicted values for the set of imaging parameters for use in acquiring further ultrasound data for a further ultrasound image.

2. The ultrasound diagnosis apparatus according to claim 1, wherein the at least one acquisition parameter comprises at least one of a wave profile parameter, an ultrasound transmission frequency, an ultrasound receiving frequency, a pulse duration, a pulse power, a frame rate, a depth parameter, a focus parameter, an F-number).

3. The ultrasound diagnosis apparatus according to claim 1, wherein at least one of a) and b):—
a) the obtaining of the predicted values for the set of imaging parameters using the imaging information comprises processing the imaging information to extract at least one feature, and supplying the at least one feature to a function defined by the training of the machine learning algorithm;
b) the obtaining of the predicted values for the set of imaging parameters using the imaging information comprises supplying the imaging information to a trained neural network.

4. The ultrasound diagnosis apparatus according to claim 1, wherein:
the processing circuitry is further configured to form a vector comprising the initial values of the set of imaging parameters; and
the obtaining of the predicted values is in dependence on the vector and on the region of interest.

5. The ultrasound diagnosis apparatus according to claim 1, wherein the processing circuitry is configured to repeat a process comprising using the predicted values to acquire ultrasound data and obtain an ultrasound image, extracting imaging information from the region of interest, and obtaining further predicted values, the process being repeated at least twice.

6. The ultrasound diagnosis apparatus according to claim 5, wherein the process is repeated until the predicted values converge.

7. The ultrasound diagnosis apparatus according to claim 5, wherein the process is repeated until a change in the predicted values falls below a threshold value continuously for a predetermined number of times.

8. The ultrasound diagnosis apparatus according to claim 1, further comprising a touch screen configured to display the ultrasound image, and receive user input representative of a location of the region of interest in the ultrasound image.

9. The ultrasound diagnosis apparatus according to claim 1, wherein the obtaining of the predicted values is performed in real time during a clinical examination.

10. A training apparatus for training a machine learning algorithm to predict values for a set of imaging parameters, the training apparatus comprising processing circuitry configured to:
for each of a plurality of anatomical regions of a plurality of subjects, obtain a user-selected set of values for a set of imaging parameters, wherein the user-selected set of values is selected by the user as providing a preferred appearance of the anatomical region of the subject in an ultrasound image;
obtain training samples for the plurality of anatomical regions of the plurality of subjects, each training sample comprising a respective set of values for the imaging parameters and an ultrasound image acquired by scanning the anatomical region of the subject using said respective set of values; and
train the machine learning algorithm using the training samples and the user-selected sets of values, such that the machine learning algorithm is configured to receive initial values for the imaging parameters and at least part of an ultrasound image obtained using the initial set of values, and to output predicted values for the imaging parameters.

11. A training apparatus according to claim 10, wherein the sets of values for the training samples are automatically generated.

12. A training apparatus according to claim 10, wherein the sets of values for the training samples are randomized.

13. A training apparatus according to claim 12, wherein the randomized sets of values for the training samples are selected in dependence on the user-selected sets of values.

14. A training apparatus according to claim 10, wherein no assessment of the quality of the training samples is provided to the machine learning algorithm.

15. A training apparatus according to claim 10, wherein the training apparatus further comprises a medical scanner configured to acquire the plurality of training samples by repeatedly scanning each anatomical region of each subject, and wherein the repeated scanning of each anatomical region is performed automatically.

16. A training apparatus according to claim 10, wherein the machine learning algorithm comprises feature-based machine learning.

17. A training apparatus according to claim 1, wherein the machine learning algorithm is based on intensity distribution and/or texture features in a user-selected region of interest.

18. A training apparatus according to claim 1, wherein training the machine learning algorithm comprises combining by a classifier at least some of: features of the ultrasound image, values for the imaging parameters, a user-selected region of interest in the ultrasound image.

19. A training apparatus according to claim 1, wherein the machine learning algorithm comprises a neural network.

20. A method for training a machine learning algorithm to predict values for a set of imaging parameters, the method comprising:
for each of a plurality of subjects,
for at least one anatomical region of the subject,
using a medical scanner to scan the anatomical region of the subject to obtain ultrasound data using at least one user-supplied set of values for a set of imaging parameters of the scanner, the set of imaging parameters comprising at least one acquisition parameter;
processing the ultrasound data to obtain an ultrasound image;
receiving a user-selected set of values that are selected by the user as providing a preferred appearance of the anatomical region of the subject in the ultrasound image;
automatically generating a plurality of sets of values for the set of imaging parameters;
using the medical scanner to scan the anatomical region of the subject using each of the automatically-generated sets of values for the set of imaging parameters, thereby to obtain for each of the automatically-generated sets of values a respective training sample, the training sample comprising the automatically-generated set of values and an ultrasound image obtained from the scanning of the anatomical region of the subject using the automatically-generated set of values; and
training a machine learning algorithm based on the training samples and user-selected sets of values, such that the machine-learning algorithm is configured to receive initial values for the imaging parameters and an ultrasound image obtained using the initial values, and to output predicted values for the imaging parameters.
